(19) United States
(12) Patent Application Publication
(10) Pub. No.: US 2015/0096734 A1
(43) Pub. Date: Apr. 9, 2015

(54) TEMPERATURE CONTROL DEVICE

(71) Applicant: MING-CHIEN CHANG, New Taipei City (TW)

(72) Inventor: MING-CHIEN CHANG, New Taipei City (TW)

(21) Appl. No.: 14/486,009

(22) Filed: Sep. 15, 2014

(30) Foreign Application Priority Data
Oct. 9, 2013 (CN) 201320620469.6

(51) Int. Cl. C12M 1/00; C12M 1/36; G01K 13/00

(52) U.S. Cl. CPC C12M 41/14; G01K 13/00; C12M 41/48
USPC 165/257; 236/3

(57) ABSTRACT

A temperature control device has a housing, a configuration unit, a microcontroller unit (MCU) and a temperature-sensing unit. After the configuration unit is used to configure a requested temperature and a temperature tolerance, the MCU adds a temperature outside the housing sensed an external temperature-sensing element of the temperature-sensing unit to the temperature tolerance, then determines if the sum is higher or lower than the requested temperature, starts a cooling unit or a heating unit to operate until a temperature inside the housing sensed by an internal temperature-sensing element of the temperature-sensing unit, thereby achieving an thermostatic effect.

### BACKGROUND OF THE INVENTION

1. Field of the Invention

The present invention is related to a temperature control device, and, more particularly, to a temperature control device providing a thermostatic effect and an improved performance on energy conservation.

2. Description of the Related Art

When conventional incubators in laboratories are used for culture of species, research personnel usually places the species to be cultivated into such a conventional incubator and then sets up an incubation temperature to be carried out (normally below 37° C.) through a configuration interface. While the conventional incubator is activated, a cooling unit and a heating unit mounted therein are simultaneously operated to maintain a temperature inside the incubator at the incubation temperature initially set up by the research personnel.

However, after the conventional incubator is configured and activated, the cooling unit and the heating unit inside the incubator need to be both started and operated. Hence, such conventional incubator inevitably consumes more power and parts of the incubator are prone to damage and repair, indicating that there is room for improvement as far as energy conservation and carbon reduction are concerned.

### SUMMARY OF THE INVENTION

The objective of the present invention is to provide a temperature control unit providing a thermostatic effect and rendering an improved performance on energy conservation and carbon reduction.

To achieve the foregoing objective, the temperature control device has a housing, an isolation plate, a guide plate, a configuration unit, a temperature-sensing unit, a microcontroller unit (MCU), a cooling unit, a heating unit and a ventilation unit.

The isolation plate is mounted inside the housing. One end of the isolation plate is connected with an inner wall of the housing.

One side of the guide plate is connected with the other end of the isolation plate for the guide plate to be isolated from the housing by the isolation plate and for an air circulation pathway to be formed inside the housing.

The configuration unit is mounted on a first outer surface of the housing, and serves to configure a temperature tolerance and a requested temperature.

The temperature-sensing unit is mounted on the housing, and serves to generate at least one sensing signal and further generate an inner measured temperature value and an outer measured temperature value.

The MCU is mounted inside the housing, receives the inner measured temperature value and the outer measured temperature value from the temperature-sensing unit and the temperature tolerance and the requested temperature from the configuration unit, and generates at least one control signal according to the inner measured temperature value, the outer measured temperature value, the temperature tolerance and the requested temperature.

The cooling unit is mounted inside the housing, receives the at least one control signal for operation, and generates a cold air.

The heating unit is mounted inside the housing, receives the at least one control signal for operation, and generates a hot air.

The ventilation unit is mounted inside the housing, receives the at least one control signal to perform a corresponding operation for the cold air and the hot air to circulate inside the housing and achieve a thermostatic effect.

From the foregoing, after the configuration unit is used to configure a requested temperature and a temperature tolerance, the MCU adds a temperature outside the housing sensed an external temperature-sensing element of the temperature-sensing unit to the temperature tolerance, then determines if the sum is higher or lower than the requested temperature, starts a cooling unit or a heating unit to operate until a temperature inside the housing sensed by an internal temperature-sensing element of the temperature-sensing unit, thereby achieving an thermostatic effect. Additionally, as only one of the cooling unit and the heating unit are operated, the temperature control device has an improved performance in terms of energy conservation and carbon reduction.

### BRIEF DESCRIPTION OF THE DRAWINGS

FIG. 1 is a cross-sectional side view a temperature control device in accordance with the present invention; and

FIG. 2 is a perspective view of the temperature control device as shown in FIG. 1.

### DETAILED DESCRIPTION OF THE INVENTION

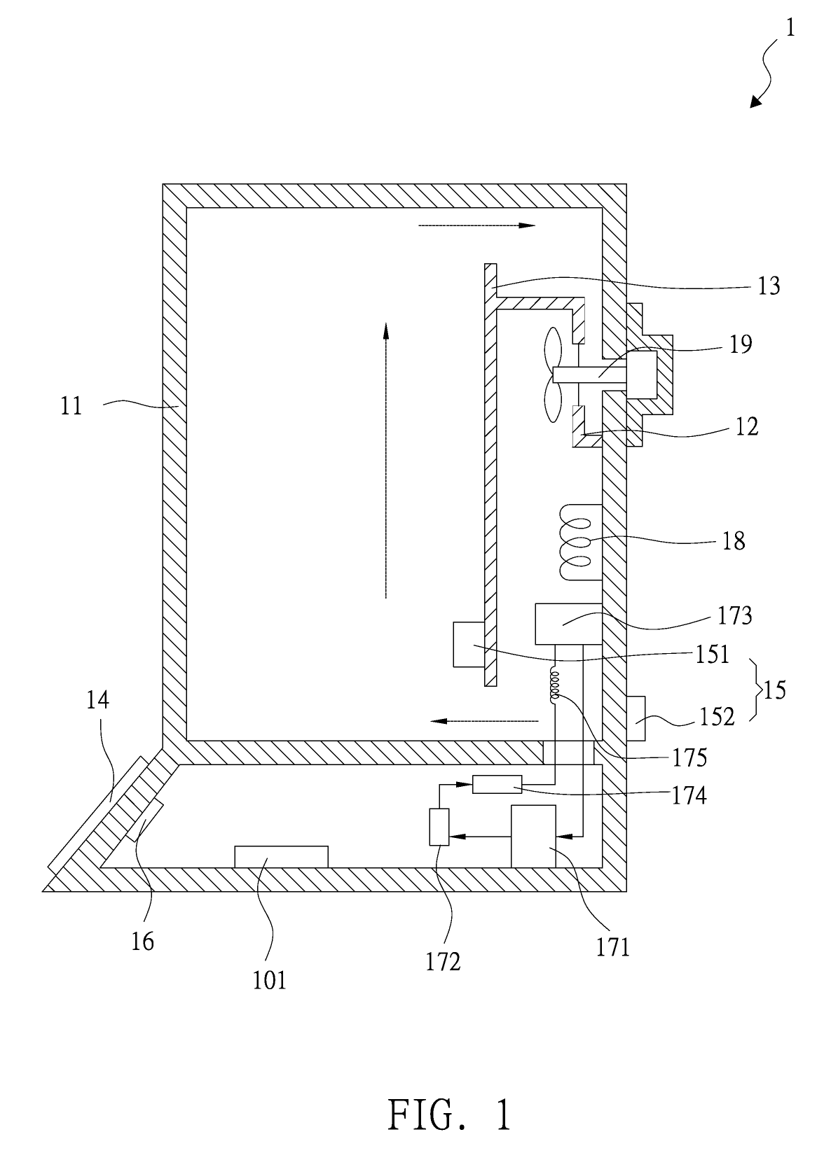

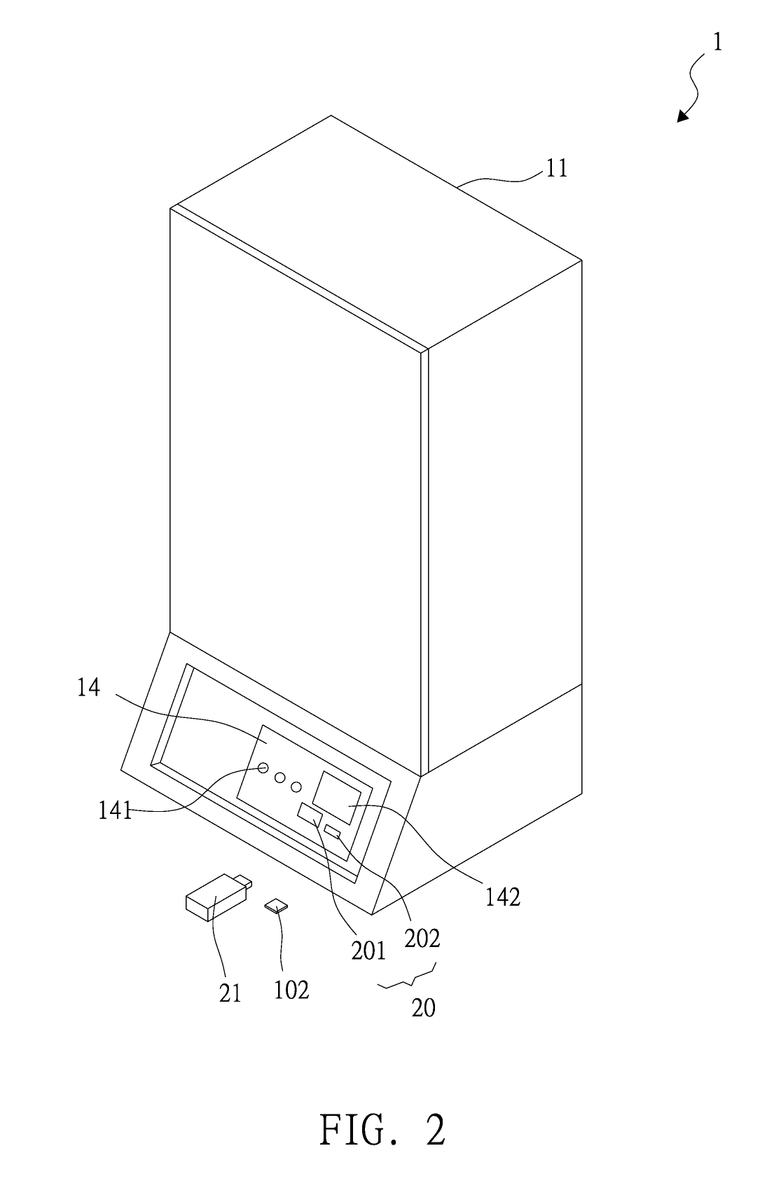

With reference to FIGS. 1 and 2, a temperature control device 1 in accordance with the present invention has a housing 11, an isolation plate 12, a guide plate 13, a configuration unit 14, a temperature-sensing unit 15, a microcontroller unit (MCU) 16, a cooling unit 17, a heating unit 18, and a ventilation unit 19.

The isolation plate 12 and the guide plate 13 are mounted inside the housing 11. One end of the isolation plate 12 is connected with an inner wall of the housing 11, and the other end of the isolation plate 12 is connected with one side of the guide plate 13, such that the guide plate 13 is isolated from the housing 11 by the isolation plate 12 and an air circulation pathway is formed inside the housing 11.

The configuration unit 14 is mounted on a first outer surface of the housing 11, serves to configure a temperature tolerance and a requested temperature, and has a configuration button 141 and a configuration panel 142.

The temperature-sensing unit 15 is mounted on the housing 11, serves to generate at least one sensing signal, and has an internal temperature-sensing element 151 and an external temperature-sensing element 152. The internal temperature-sensing element 151 is mounted on the other side of the guide plate 13 opposite to the isolation plate 12 to sense a temperature inside the housing 11, and generate an internal sensing signal and an inner measured temperature value. The external temperature-sensing element 152 is mounted on a second outer surface of the housing 11 to sense a temperature outside the housing 11, and generate an external sensing signal and an outer measured temperature value.

The MCU 16 is mounted inside the housing 11, receives the inner measured temperature value, the outer measured temperature value, the temperature tolerance and the requested temperature, and generates at least one control signal according to the inner measured temperature value, the outer measured temperature value, the temperature tolerance and the requested temperature.

The cooling unit 17 is mounted inside the housing 11, receives the at least one control signal to perform a corresponding operation, and generates a cold air. The cooling unit 17 has a compressor 171, a condenser 172, an evaporator 173, a dryer 174 and a throttle 175.

The heating unit 18 is mounted inside the housing 11, receives the at least one control signal to perform a corresponding operation, and generates a hot air.

The ventilation unit 19 is mounted inside the housing 11, and receives the at least one control signal to perform a corresponding operation for the cold air and the hot air to circulate inside the housing 11 and achieve a thermostatic effect.

When research personnel sets the temperature tolerance at 3° C. and the requested temperature inside the housing 11 at 24° C. through the configuration button 141 of the configuration unit 14. The MCU 16 receives the temperature tolerance and the requested temperature. The temperature-sensing unit 15 senses temperatures inside and outside the housing 11. The external temperature-sensing element 152 of the temperature-sensing unit 15 senses the temperature outside the housing 11 at 26° C., and transmits an outer sensing signal that is generated from sensing the temperature outside the housing 11 to the MCU 16. The MCU 16 further adds the temperature outside the housing 26° C. to the temperature tolerance 3° C. to obtain a temperature total at 29° C., and compares the temperature total with the requested temperature 24° C. As determining that the temperature total is 5° C. higher than the requested temperature (first determination result), the MCU 16 generates a first control signal, which is an enable signal for cooling, to start the cooling unit 17 for operation in generation of the cold air. Because the ventilation unit 19 gets started when the temperature control device 1 is activated, the ventilation unit 19 circulates the cold air inside the housing 11 to lower the temperature inside the housing 11. Moreover, while the temperature inside the housing 11 is lowered by the cooling unit 17, the internal temperature-sensing element 151 inside the housing 11 simultaneously senses the temperature inside the housing 11. When the temperature inside the housing 11 sensed by the internal temperature-sensing element 151 approaches the requested temperature 24° C. configured by the research personnel, the internal temperature-sensing element 151 will send an inner sensing signal that is generated from sensing the temperature inside the housing to the MCU 16. The MCU 16 then determines if the temperature inside the housing 11 needs to be lowered so as to approach the requested temperature 24° C. according to the inner sensing signal transmitted from the internal temperature-sensing element 151 (second determination result). If the temperature sensed inside the housing 11 approaches the requested temperature 24° C., the MCU 16 generates a second control signal, which is an enable signal for heating, to start the heating unit 18 for operation in generation of the hot air. The ventilation unit 19 circulates the hot air inside the housing 11 to maintain the temperature inside the housing 11 at the requested temperature 24° C. configured by the research personnel so that the temperature control device 1 achieves a requested thermostatic effect.

6 On the other hand, suppose that the research personnel configures the temperature tolerance at 1° C. and the requested temperature inside the housing 11 at 28° C. and activates the temperature control device 1 for operation. When the external temperature-sensing element 152 senses the temperature outside the housing 11 at 23° C. and transmits the sensing signal that is generated from sensing the temperature outside the housing 11 to the MCU 16. The MCU 16 further adds the temperature 23° C. sensed by the external temperature-sensing element 152 to the temperature tolerance to obtain a temperature total at 24° C., and then compares the temperature total with the requested temperature 28° C. As determining that the temperature total is lower than the requested temperature, the MCU 16 generates the second control signal, which is the enable signal for heating, to start the heating unit 18 for operation. The ventilation unit 19 circulates the hot air generated by the heating unit 18 inside the housing 11 to achieve a temperature-rising effect inside the housing 11 so that the temperature inside the housing can be maintained at the requested temperature 28° C. configured by the research personnel and maintain a thermostatic effect on the temperature inside the housing 11. The requested temperature is adjustable depending on the characteristics of the species to be studied or to be cultivated, and includes but is not limited to the requested temperatures configured as given in the foregoing description. Similarly, the temperature outside the housing 11 relies on an actual detection, and is not limited to the temperature outside the housing 11 sensed as given in the foregoing description. The temperature tolerance can be also slightly adjusted based on operational and experimental requirements, and is not limited to the temperature tolerance given in the foregoing description.

With further reference to FIG. 2, the inner measured temperature value and the outer measured temperature value sensed by the temperature control device 1 inside and outside the housing 1, the configured temperature tolerance and the requested temperature are displayed on the configuration panel 14.

The temperature control device 1 further has a storage unit 10 and an output unit 20. The storage unit 10 is used for the MCU 16 to store the inner measured temperature value sensed by the internal temperature-sensing element 161, and may be a hard disk 101 or a memory card 102 as shown in FIGS. 1 and 2.

The output unit 20 is electrically connected to a portable storage device 21 to store the inner measured temperature value transmitted from the storage unit 10 to the portable storage device 21, and may be a universal serial bus (USB) interface 201 or a card reader 202.

Accordingly, the temperature control device 1 of the present invention has the following advantages over the conventional incubator.

1. After the temperature control device 1 configures the requested temperature and the temperature tolerance through the configuration unit 1, the MCU 16 determines if a temperature total obtained by summing the outer measured temperature value sensed by the external temperature-sensing element 152 of the temperature-sensing unit 15 and a temperature tolerance is higher or lower than the requested temperature, and only starts the cooling unit 17 for operation if the temperature total is higher than the requested temperature or only starts the heating unit 18 for operation if the temperature total is lower than the requested temperature until the temperature measured inside the housing 11 approach the requested temperature. Accordingly, the present invention can achieve a thermostatic effect and improve the drawback of being more power-consuming and failure-prone.

2. The temperature control device is applied to culture of animal and plant cells and of microbiologic and biochemical experiments to attain the goal of energy conservation, carbon reduction and thermostatic operation.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A temperature control device, comprising:
a housing;
an isolation plate mounted inside the housing, wherein one end of the isolation plate is connected with an inner wall of the housing;
a guide plate, wherein one side of the guide plate is connected with the other end of the isolation plate for the guide plate to be isolated from the housing by the isolation plate and for an air circulation pathway to be formed inside the housing;
a configuration unit mounted on a first outer surface of the housing, and serving to configure a temperature tolerance and a requested temperature;
a temperature-sensing unit mounted on the housing, and serving to generate at least one sensing signal and further generate an inner measured temperature value and an outer measured temperature value;
a microcontroller unit (MCU) mounted inside the housing, receiving the inner measured temperature value and the outer measured temperature value from the temperature-sensing unit, and the temperature tolerance and the requested temperature from the configuration unit, and generating at least one control signal according to the inner measured temperature value, the outer measured temperature value, the temperature tolerance and the requested temperature;
a cooling unit mounted inside the housing, receiving the at least one control signal for operation, and generating a cold air;
a heating unit mounted inside the housing, receiving the at least one control signal for operation, and generating a hot air; and
a ventilation unit mounted inside the housing, receiving the at least one control signal to perform a corresponding operation for the cold air and the hot air to circulate inside the housing and achieve a thermostatic effect.

2. The temperature control device as claimed in claim 1, wherein the configuration unit has:
a configuration panel serving to display the inner measured temperature value, the outer measured temperature value, the configured temperature tolerance and the requested temperature; and
a configuration button serving to set the temperature tolerance and the requested temperature and control the temperature control device through the MCU.

3. The temperature control device as claimed in claim 1, wherein the temperature-sensing unit has an internal temperature-sensing element and an external temperature-sensing element.

4. The temperature control device as claimed in claim 3, wherein the internal temperature-sensing element is mounted inside the housing, senses a temperature inside the housing, generates an internal sensing signal, and sends the internal sensing signal to the MCU for the MCU to generate a first determination result and the at least one control signal according to the first determination result; and
the external temperature-sensing element is mounted on an outside of the housing, senses a temperature outside the housing and generate an external sensing signal, generates an external sensing signal, and sends the external sensing signal to the MCU for the MCU to generate a second determination result and the at least one control signal according to the second determination result.

5. The temperature control device as claimed in claim 1, wherein the cooling unit has a compressor, a condenser, an evaporator, a dryer and a throttle.
